(19) United States
(12) Patent Application Publication
(10) Pub. No.: US 2023/0346994 A1
(43) Pub. Date: Nov. 2, 2023

(54) ELECTROCEUTICAL FABRIC FOR ERADICATING CORONAVIRUS

(71) Applicant: THE TRUSTEES OF INDIANA UNIVERSITY, Bloomington, IN (US)

(72) Inventors: Chandan K. SEN, Indianapolis, IN (US); Sashwati ROY, Indianapolis, IN (US); Subhadip GHATAK, Indianapolis, IN (US); Savita KHANNA, Indianapolis, IN (US)

(21) Appl. No.: 17/923,051

(22) PCT Filed: May 11, 2021
(86) PCT No.: PCT/US2021/031683

§ 371 Date: Nov. 3, 2022

Related U.S. Application Data
(60) Provisional application No. 63/024,164, filed on May 13, 2020.

(51) Int. Cl. A61L 2/03

(52) U.S. Cl. CPC A61L 2/03; A61L 2209/14; A61L 2202/26; A61L 2300/102

(57) ABSTRACT

Compositions and methods are provided for preparing an article that comprises an electroceutical material that comprises a redox pair of alternating metals that generate an electric current or field when exposed to an aqueous solution. In one embodiment filtering equipment including facial masks are prepared comprising the electroceutical materials.

### CROSS REFERENCE TO RELATED APPLICATIONS

This application claims priority to the following: U.S. Provisional Patent Application No. 63/024,164 filed on May 13, 2020, the disclosure of which is expressly incorporated herein.

### BACKGROUND OF THE DISCLOSURE

Coronavirus Disease 2019 (COVID-19) has been confirmed worldwide and was declared a pandemic in March of 2020 by the World Health Organization (WHO). SARS-CoV-2 infection causes COVID-19. The basic reproductive number of an infection, denoted as Ro, gauges the number of susceptible individual(s) that an infectious host can spread their disease to. While this epidemiological metric for severe acute respiratory syndrome (SARS) was reported to be 3 by the WHO, recent studies on COVID-19 estimates its Ro to be 5.7 making containment more challenging. Statistically, aplon infection becomes self-limiting when the Ro drops below 1 such as when a population acquires herd immunity. Respiratory infections are known to spread through direct routes of aerosolization such as sneezing, coughing and other contact gestures. In addition, indirect modes of transmission play a significant role in determining Ro.

SARS-CoV-2 remains viable for an extended period of time. Strikingly, the Center for Disease Control and Prevention (CDC) of the United States reported presence of SARS-CoV-2 RNA on various surfaces on the Diamond Princess ship 17 days after all symptomatic and asymptomatic COVID-19 passengers had vacated. In a laboratory-based experimental study, SARS-CoV-2 remained viable for at least three hours in aerosols and up to 72 hours on fomites such as stainless steel. Such persistence of infectious virus particles in the environment increases the probability of spreading infection, thereby increasing Ro. While social distancing and quarantine measures break direct chains of transmission of the disease, viable virus particles in the surroundings of the person contribute to the spread of COVID-19. Use of personal protective equipment (PPE) is essential to safeguard healthcare providers against COVID-19. However, use of these PPE itself poses significant threat as doffing of contaminated PPE carrying viable viral particles is likely to infect the person and potentially spread infection. Although CDC has recommended strict procedures for removing personal protective equipment (PPE), to reduce risk of nosocomial infections, contaminated PPE poses an imminent serious risk for healthcare professionals. A need exists to provide PPE that can eliminate the virus itself to reduce the spread.

### SUMMARY

The present disclosure is directed to a method of using chemoelectrical intervention (CEI) to inactivate viruses, where energy in the form of electric field or current, provides a therapeutic and/or prophylactic effect, to render viral pathogens less pathogenic. For example, the CEI may reduce the number of infectious agents below a threshold required for infectivity or may otherwise render the viral pathogen less pathogenic. This principle applies across multiple viral pathogens, including RNA viruses that are of relevance to human disease such as influenza and coronavirus.

The present disclosure employs a novel biophysical approach that has efficacy on its own and may complement conventional biopharmaceutical approaches to treat and manage infections. In accordance with one embodiment the method exerts a relatively weak electric field or current of specific strength, ranging from 5 V/cm to 50 V/cm, 0.5 V/cm to 5 V/cm, 0.5 V/cm to 1 V/cm or about 0.25 V/cm to about 1 20 v/cm, sufficient to manage risk of infection by providing at least microbial static effects, i.e., blocking their multiplication, and in some cases providing microbial cidal effects, i.e., killing them. In one non-limiting embodiment, the method applies an electric field or current to which the microbes are sensitive using a wireless electroceutical device or dressing (WED).

In one embodiment the electroceutical device comprises a fabric of synthetic fibers that comprises a pattern of alternating metals that form an appropriate oxidation-reduction (redox) couple for generating an electric field or current due to the transfer of an electron from one metal to the other when contacted with an aqueous solution, and in the absence of a power source. In one embodiment the electroceutical device comprises alternating dots of silver and zinc as the redox couple. In one embodiment the electroceutical device comprises a fabric of synthetic fibers having Ag dots (1-2 mm) and Zn dots (1-2 mm) printed on the fabric in proximity of about 0.5, 1, 1.5 or 2 mm to each other. In one embodiment the electroceutical device comprises a fabric of synthetic fibers having Ag dots (of about 2 mm) and Zn dots (of about 1 mm) printed on the fabric in proximity of about 0.5, 1, or 1.5 to each other. In one embodiment the electroceutical device comprises an FDA approved silver-zinc coupled bioelectric dressing (BED) which is currently being used in clinical wound care. Such a device is commercially available from Vomaris Inc.

(Phoenix, AZ). The advantage of this device is that it is wireless and has no need for an external power source, can be cut to any desired shape and size, conforms to irregular surfaces, and provides an electrical field in the range of the physiologic fields (Banerjee et al, PLoS Onev.9(3); 2014).

In accordance with one embodiment a method of inactivating an RNA virus is provided. The method comprises exposition the virus to a weak electrical field or current for about 0.5, 1, 2, 3 or 5 minutes. In one embodiment the RNA virus is a corona, Ebola, severe acute respiratory syndrome (SARS), or influenza virus. In one embodiment the RNA virus is a corona or influenza virus and in one embodiment the virus is a corona virus, including for example COVID19.

In one embodiment the electrical field is generated using a wireless device that comprises alternating spots of a redox pair of metals, including for example silver and zinc. In one embodiment the virus is exposed to a potential difference of about 0.25 to about 1.0 V. In accordance with one embodiment the method of inactivating an RNA virus comprises contacting the virus with an electroceutical material in the presence of an aqueous solution, wherein the electroceutical material comprises a plurality of silver and zinc regions that are imprinted on said material and are spaced from each other at a distance that upon contact with said aqueous solution generates an electric field or current. In one embodiment the virus comes in contact with electroceutical material when air comprising said virus is directed or drawn across, or through, said electroceutical material in the presence of an aqueous solution, optionally wherein the electroceutical material is part of a filtration device. In accordance with one embodiment the filtration device comprises an electroceutical material wherein the material is formed as a fabric of synthetic fibers, wherein the fabric has been imprinted with 1-2 mm sized particles of silver and zinc printed in an alternating pattern on the fabric within about 0.5 to 1.5 mm proximity of each other. In one embodiment the electroceutical material generates a potential difference of about 0.25 to about 1 V/cm, or about 0.5 V/cm upon contact of the electroceutical material with an aqueous solution.

In one embodiment the synthetic fibers forming the electroceutical fabric are elastic, optionally wherein the synthetic fibers comprises a polymer selected from the group consisting of polyurethane, polytrimethylene terephthalate and polybutylene terephthalate fiber. In one embodiment, the electroceutical material is a component of a facial mask or screen and the target virus for elimination is a coronavirus. IN one embodiment the facial mask is a non-woven fabric comprised of polypropylene, optionally at 20 or 25 grams per square meter.

In accordance with one embodiment an air filtration system for reducing the spread of an RNA virus is provided, wherein the system comprises an electroceutical material. In one embodiment the electroceutical material comprises a material having a plurality of silver and zinc regions imprinted on a surface of the material and spaced from each other at a distance that upon contact with an aqueous solution generates an electric field or current that disrupts the electrokinetic properties of a virus placed in contact with the electrical field generated by the electroceutical material. In one embodiment the target virus is a coronavirus. In one embodiment the air filtration system is a component of an article of personal protective equipment, optionally wherein the personal protective equipment is a face mask, optionally wherein alternating silver and zinc spots are imprinted on the outer exposed surface of the facemask at a distance from each other such that upon contact with said aqueous solution an electric field or current is generated that disrupts the electrokinetic properties of a virus placed in contact with the electrical field generated by the electroceutical material. In one embodiment the generated electrical field results from a potential difference of about 0.25 to about 1.0 V between opposing redox pairs.

### BRIEF DESCRIPTION OF THE DRAWINGS

FIGS. 1A-1C: Deposition of Ag and Zn on a fabric used for face-mask production was tested by EDX spectrum analysis for generation of an electric potential in the presence of three ionized aqueous media. The potential difference between adjacent Ag and Zn deposits in an NaCl solution (FIG. 1A; 0.85% w/v), cell culture medium (FIG. 1B) and tap water (FIG. 1C: of practical value to end users of PPE) were tested at room temperature. The potential difference between the two electrodes rapidly increased and achieved a steady state after the first 15 s.

FIG. 2A shows absolute quantification of viral particles recovered from the fabric after treatment with fₑ. A two-fold and four-fold reduction in the recovered viral number was observed after 1 minute (fₑ1) and 5 (fₑ5) minute treatment, respectively.

FIG. 2B shows changes in viral zeta potential and affiliated parameters after contact with the fabric for 1 min or 5 min. Lane 1: 10⁵ stock applied CoV; Lane 2: 4 ×10⁴ recovered CoV after contact with the fabric for 1 min; and Lane 3: 2.5×10⁴ recovered CoV after contact with the fabric for 5 min. Data are mean±SEM.

FIG. 3A is a bar graph presenting quantitative plotting of changes in cell viability as determined by PI/calcein expressed as fold-change over the basal cell death level expected as part of standard cell culture process. Infected cells were monitored for appearance of cytopathic effects (CPE; cell rounding and sloughing) until post-infection day 7 after ST cells were infected with viruses pre-exposed to the electroceutical fabric for 1 or 5 minutes, respectively (fₑ1 and fₑ5) or to sham control fabric for 1 or 5 minutes, respectively (fₛ1 and fₛ5). ST cells infected with untreated virus or fₛ-contacted virus showed distinct signs of CPE and loss of cell viability. Cells infected virus which were subjected to contact with the electroceutical fabric for 1 (fₑ1) or 5 (f_{c}5) minute did not display any further loss of cell viability above and beyond the basal level of cell death expected at that phase of the life-cycle of the cell.

FIG. 3B is a bar graph presenting changes in cell viability as determined by the MTT (3-(4,5-dimethylthiazol-2-yl)-2,5-diphenyltetrazolium bromide) assay. The cytopathic effects of CoV and the protective effects of electroceutical fabric (fₑ) (versus sham electroceutical fabric (fₛ)) was corroborated by the standard MTT assay commonly used for testing cell viability.

FIG. 4 is a bar graph presenting data from mammalian cells treated with purified lentivirus or the same virus subjected to contact with electroceutical fabric (fₑ) or sham electroceutical fabric (fₛ) for 1 or 5 mins as indicated. Recovered viral particles (4×10⁴) were counted and used to treat cells at MOI of 10. After 96 h of infection, HEK293 cells were microscopically assessed for the expression of GFP which would be an endpoint of successful infection. Treatment of cells with virus recovered from sham fabric fₛ caused comparable infection (FIG. 4). However, contact of virus with the electroceutical fabric fₑ, even for one minute, eliminated lentiviral infectivity (FIG. 4). Data are mean±SEM.

### DETAILED DESCRIPTION

### Definitions

In describing and claiming the invention, the following terminology will be used in accordance with the definitions set forth below.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

As used herein, the term “treating” includes prophylaxis of the specific disorder or condition, or alleviation of the symptoms associated with a specific disorder or condition and/or preventing or eliminating said symptoms.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified RNA” is used herein to describe an RNA sequence which has been separated from other compounds including, but not limited to polypeptides, lipids and carbohydrates.

The term “isolated” requires that the referenced material be removed from its original environment (e.g., the natural environment if it is naturally occurring). For example, a naturally-occurring nucleic acid present in a living animal is not isolated, but the same nucleic acid, separated from some or all of the coexisting materials in the natural system, is isolated.

As used herein the term “patient” without further designation is intended to encompass any warm blooded vertebrate domesticated animal (including for example, but not limited to livestock, horses, mice, cats, dogs and other pets) and humans receiving a therapeutic treatment whether or not under the direct care of a physician.

As used herein the term “solid support” relates to a solvent insoluble substrate that is capable of forming linkages (preferably covalent bonds) with soluble molecules. The support can be either biological in nature, such as, without limitation, a cell or bacteriophage particle, or synthetic, such as, without limitation, an acrylamide derivative, glass, plastic, agarose, cellulose, nylon, silica, or magnetized particles. The support can be in particulate form or a monolythic strip or sheet. The surface of such supports may be solid or porous and of any convenient shape.

As used herein, the term “parenteral” includes administration subcutaneously, intravenously or intramuscularly.

As used herein the term “nuclease” is defined as any enzyme that can cleave the phosphodiester bonds between nucleotides of nucleic acids. The term encompasses both DNases and RNases that effect single or double stranded breaks in their target molecules. A DNase is a nuclease that catalyzes the hydrolytic cleavage of phosphodiester linkages in a DNA backbone, whereas an RNase is a nuclease that catalyzes the hydrolytic cleavage of phosphodiester linkages in an RNA backbone. The nuclease may be indiscriminate about the DNA sequence at which it cuts or alternatively, the nuclease may be sequence-specific. The nuclease may cleave only double-stranded nucleic acid, only single-stranded nucleic acid, or both double-stranded and single stranded nucleic acid. The nuclease can be an exonuclease, that cleaves nucleotides one at a time from the end of a polynucleotide chain or an endonuclease that cleaves a phosphodiester bond within a polynucleotide chain. Deoxyribonuclease I (DNase I) is an example of a DNA endonuclease that cleaves DNA (causing a double stand break) relatively nonspecifically in DNA sequences.

As used herein an antimicrobial is any agent that kills microorganisms or stops their growth, including microorganisms selected from the group consisting of bacteria, protists, and fungi.

As used herein the term “adversely affecting” a virus means that the viability and/or infectivity of the virus is compromised in some way. For example, a virus will be adversely affected if the number of infectious particles in a sample is reduced after treatment.

As used herein the expression “effective amount” refers to the amount of a substance sufficient to achieve a desired result, which in the case of the present invention is to adversely affect a virus. The exact amount required to achieve the desired result will vary depending on various factors such as the viral strain, or the age or condition of the target host subject. Accordingly, it is not practical to specify an exact “effective amount”. Taking into account the particular circumstances, a person skilled in the art could readily determine the “effective amount” through routine experimentation.

As used herein an “RNA virus” is a virus that has ribonucleic acid (RNA) as its genetic material. This nucleic acid is usually single-stranded RNA (ssRNA) but may be double-stranded RNA (dsRNA).

As used herein the abbreviations “WED” (for wireless electroceutical device) and “BED” (for bioelectric dressing) are used interchangeably and in the absence of further characterization define any wireless electroceutical dressing or device that delivers electrical stimulation to biological tissues upon contact of those tissues in the presence of an aqueous solution.

### EMBODIMENTS

Infectivity of a viral particle is dependent on its stability and determines its ability to spread infection. Multiple biophysical factors determine the stability of coronaviruses, and viruses in general, both within and outside the host. For instance, nonspecific electrostatic interactions influence capsid assembly of enveloped RNA viruses in which positively charged capsid proteins package the negatively charged RNA. In positive-sense single-stranded RNA viruses, such as the coronaviruses, this thermodynamically spontaneous assembly is mediated by arginine rich motifs.

It has been recently discovered that a large number of ss-RNA-viruses follow a general law of packaging, based only on electrostatic forces without an explicit dependence on the sequence specificity. The crystallized RNA portions of these single-stranded RNA viruses are comprised of A-type double-stranded helical RNA. These ds helical RNA are formed by intrachain H-bonding of self-complementary RNA sequences. Therefore, although the complex formation between RNA with the inner capsid surface is independent of RNA sequence, the complexed RNA itself is made up of a helical secondary structure associated with sequence-matched base pairing. The overall positive charge of the capsid also limits viral genome length. However, coronaviruses express an exoribonuclease associated with nonstructural protein 14 which allows them to inherit longer genomes when compared to other RNA viruses.

Destabilization of coronavirus and other ssRNA viruses outside the host is of paramount importance in abating the spread of infection. The effectiveness of electroceutical principles as an alternative to pharmacological approaches in managing planktonic microbial pathogens and complex polymicrobial biofilms has been established by the applicant. In accordance with the present disclosure such techniques are now used to control the spread of pathogenic viruses including coronaviruses. More particularly, the present disclosure provides compositions and methods for combatting the spread of viral pathogens including rotavirus, norovirus, influenza and coronavirus and other ssRNA viruses through the use of an electroceutical material. In one embodiment, a method of eradicating or reducing the number of infectious viral particles associated with a surface is provided. In one embodiment, a method of eliminating or reducing the infectivity of a coronavirus or other ssRNA virus is provided, wherein the virus is exposed to a low voltage (0.25-1.5 v/cm) electrical field. In one embodiment, a wireless electroceutical fabric is used in a method of disrupting the electrokinetic properties of the coronavirus.

Recently applicant has co-developed a wireless electroceutical fabric for use as a wound care dressing (See Banerjee et al, PLoS One 2014, 9:e89239 and Ghatak et al, Adv Wound Care (New Rochelle) 2015; 4: 302-311, the disclosures of which are expressly incorporated herein). This dressing, upon contact with bodily fluids or other aqueous wetting media, generates weak electric field which is effective in managing biofilm infection and improving wound healing. The dressing is currently FDA cleared and in clinical trial.

All charged constituents within the virus nanoenvironment are arranged in an electrostatic field which can be defined by the Poisson-Boltzmann equation, and therefore may be altered by an external electric field. In accordance with one embodiment application of a weak electrical field, for example through the use of an electroceutical fabric, is used to curb coronavirus infectivity. In one embodiment personal protective equipment comprising electroceutical fabric is provided. The electroceutical fabric of the present disclosure is simple in configuration does not require any complex wiring or power source. It is thus easy to use in a field setting and requires no training or skills. The textile itself is comparable to any other standard textile lending itself for manufacturing of PPE. Viruses are known to rely on electrostatic interactions for optimal virion assembly and attachment. For instance, structural proteins in coronaviruses, negatively charged amino acid residues in the nucleocapsid facilitates assembly with the membrane protein. Additionally, the coronavirus envelope protein is known to generate ion conductive pores across membranes which are voltage dependent. Leveraging these viral characteristics to achieve viral inactivation represents a novel approach for combating viral pathogens.

The present disclosure presents the first evidence demonstrating that the infectivity of the CoV may be disrupted using a simple electroceutical fabric. In accordance with one embodiment the electroceutical fabric of the present disclosure comprises silver and zinc dots on polyester fabric that forms a redox couple (see Example 14 for details). However, other combinations of metals beyond Ag—Zn that form an appropriate redox couple can also be used to form an electroceutical fabric suitable for use in accordance with the present disclosure.

As disclosed herein coronavirus (CoV) infectivity may be rapidly eradicated upon contact with the electroceutical fabric. Zeta potential of a particle determines its electrostatic interactions in particle dispersions and, as such, is an important determinant of the stability of viral particles. Contact of CoV with an electroceutical fabric of the present disclosure rapidly lowered the zeta potential demonstrating a direct effect of the fabric on the electrokinetic properties of the viral particle. Any change of zeta potential towards zero is an indication of an increase in electrical instability of the particle. The observation that contact with the electroceutical fabric eliminates infectivity of the virus leads to the hypothesis that the observed lowering of zeta potential may have caused defects in the structural integrity of the virus.

CoV is a nanoparticle. Nanoparticle tracking analysis determines the hydrodynamic diameter of the analyte by applying the Stokes-Einstein equation after measuring the Brownian motion of individual nanoparticle. It is an alternative method to dynamic light scattering which utilizes the same principle and is validated for assessing polydispersity and purity in viral vaccine preparations. NTA was therefore utilized to estimate absolute viral particle number and size distribution in not only pure CoV but also in CoV recovered from the fabric. Observed changes in particle number and size distribution support the aforementioned hypothesis that exposure to the weak electric field causes damaging structural alterations to the virions. Cells in culture routinely display a small fraction of dead or dying cells. Cytopathic effects of viral infection are tested to examine whether exposure to the infectious particle adds to the basal cell death burden of the culture. Long-term observations, i.e., days versus hours, ensure the recording of the eventual fate of the affected cells. Reporting of short-term data alone, while sometimes may be encouraging with respect to effect of the intervention, may simply reflect results representing postponement of death from the insult and not a true rescue. In CPE studies of this work, cell rounding and sloughing were evident in day 4 post-infection. During this time, cells treated with virus pre-exposed to the electroceutical fabric closely resembled cells that were unchallenged by exposure to the virus. In standard cell culture, the growth medium is changed every other day to wash off floating dead cells and to replenish nutrition.

Under conditions of infection by virus, such frequent change of cell culture medium is not made. Cells grow in the same spent media until day 7 post-infection. Maintenance of cells without any change in culture media for seven days is expected to marginally increase basal cell death burden as shown.

Textiles evaluated for use in PPE such as masks are subject to specific FDA 510(k) requirements expecting stringent viral filtration tests to demonstrate 99.9% reduction of 1.1-3.3×10⁴ plaque forming units of standard phiX174 bacteriophage. The phiX174 is widely used as a model organism because of it being a standardized test. However, it is important to note that unlike SAR-CoV-2 which is an RNA virus, phiX174 bacteriophage is a DNA virus with numerous contrasting physical, chemical as well as biological properties. Furthermore, this bacteriophage is much smaller in size than SAR-CoV-2. The non-enveloped icosahedral morphology of phiX174 bacteriophage aerosolizes with a mean particle size of 3.0±0.3 μm. This is in direct contrast with the coronaviruses that cause diseases in animals and humans which are ˜100 nm in diameter and are aerosolized as respiratory droplets with sizes >5 μm. Importantly, phiX174 cannot infect mammalian cells. It infects and forms visible plaques on a lawn of Escherichia coli (Migula) Castellani and Chalmers strains. In the context of COVID-19 pandemic, our study focuses on RNA viruses such as coronavirus and tests cytopathic effects on mammalian cells. Testing methods such as AATCC TM100 recommends a textile contact time of 24 h for both enveloped and non-enveloped viruses. We report results on contact time that is much shorter and more relevant to PPE usage in the context of COVID-19. Additional studies in our laboratory show effective neutralization of a wider range of viruses at a much higher load (10⁸) within about 1 to 2 hours of textile contact time (not shown).

This work presents evidence demonstrating that the physical characteristic features of CoV may be exploited to render it non-infective following exposure to weak electric field generating electroceutical fabric. The effect is rapid and achieved within one minute of contact. The observation that lentiviral infectivity is also eliminated following contact with the electroceutical fabric contributes to the rigor of our central finding. Lowering of zeta potential of the CoV particles following exposure to the electroceutical fabric constitutes direct evidence supporting that electrokinetic stability of the viral particle is weakened.

One embodiment of the present disclosure is directed to a method of manufacturing an electroceutical fabric that is useful for eliminating coronavirus. In one embodiment the electroceutical fabric is incorporated into PPE as a method of reducing the spread of coronavirus and/or disrupting the infectivity of coronavirus.

In one embodiment, a facemask comprising an electroceutical fabric is provided for eliminating coronavirus. In one embodiment, the fabric is designed to eliminate the coronavirus by lowering the zeta potential of the virus resulting in a destabilization of its electokinetic properties.

Coronavirus with intact infectivity that is attached to the PPE surfaces pose significant threat to the spread of COVID-19. Electrostatic forces determine the structure and function of coronaviruses with characteristic positively-charged capsid and negatively-charged single-stranded RNA. As disclosed herein the electroceutical fabric disrupts the infectivity of coronavirus upon contact by destabilizing the electrokinetic properties of the viral particle.

A respiratory coronavirus (USDA permit 141794) and the corresponding mammalian ST cell were obtained from ATCC to study the cytopathic effects of viral infection. Viral particles (10⁵) were placed in direct contact with the electroceutical or sham fabric for either 1 or 5 minutes. Viral particles (4×10⁴) were recovered from the fabric and subjected to nanoparticle tracking analysis and measurement of zeta potential. Recovered viral particles were subjected to cytopathic testing and studied for 7 days following infection. Under conditions of cytopathic testing, the electroceutical fabric generated a weak potential difference of 0.5V. Following one minute of contact, zeta potential of the coronavirus was significantly lowered indicating destabilization of its electrokinetic properties. Size-distribution plot showed appearance of aggregation of the virus. Testing of the cytopathic effects of the virus showed eradication of infectivity as quantitatively assessed by calcein-PI and MTT cell viability tests. This work presents first evidence demonstrating that the physical characteristic features of CoV may be exploited to render it non-infective following exposure to weak electric field generating electroceutical fabric. The effect is rapid and achieved within one minute of contact. The supporting observation that lentiviral infectivity is also eliminated following contact with the electroceutical fabric contributes to the rigor of our central finding. This work provides compelling evidence to consider electroceutical fabrics as material of choice for the development of PPE in the fight against RNA based viruses such as COVID-19.

One embodiment of the present disclosure is directed to a method of reducing the infectivity or reducing resident viral populations in an article or material that is susceptible to contamination by pathogenic viruses. In one embodiment the method comprises a step of applying a predetermined optimized electric current or field to the virus coming in contact with the article.

In accordance with one embodiment a method of managing the risk of infections from an article or material that is susceptible to contamination by a viral pathogen is provided, wherein the method comprises providing a predetermined optimized electric current or field to the article or material.

In accordance with one embodiment a method of disrupting the electrokinetic properties of an RNA virus, optionally a ssRNA virus such as coronavirus is provided. The method, in one embodiment comprises contacting the virus with an electric field, optionally generated by an electroceutical fabric in the presence of an aqueous solution, wherein the electroceutical material comprises a plurality of silver and zinc regions that are imprinted on said material and are spaced from each other at a distance that upon contact with said aqueous solution generates an electric field or current that disrupts the electrokinetic properties of a the RNA virus. In one embodiment the RNA virus is a coronavirus wherein the coronavirus is exposed to an electric field having a potential difference of about 0.25 to about 1.0 V/cm.

### EXAMPLES

The following Examples are provided to expand and further support the above disclosed embodiments. The Examples are not meant to be limiting. Further, any reference or patent application mentioned in this specification are each hereby incorporated in their entirety.

### Example 1: Electroceutical Fabric

An FDA cleared wireless electroceutical dressing was used as a source of weak electric field for the current study and is referred to as electroceutical fabric (fₑ). This fabric, co-developed by our laboratory, has been commercialized by Vomaris Inc. (Phoenix, AZ) was provided to us by the manufacturer. It is made of polyester fabric printed with alternating circular dots of Ag and Zn metals (˜2 mm and 1 mm, respectively), generating electric fields. A polyester fabric without any metal deposition (hence unable to generate electric field) was used as an experimental control and is referred to as sham fabric (fₛ).

### Example 2: Viruses and Cell Lines

Respiratory coronavirus (ATCC® VR2384™) and its host porcine cell line-ST (ATCC® CRL-1746™), recommended for its infection and propagation, were procured from ATCC (Manassas, VA). Lentivirus—CSCGW mut6 [HIV-1-based vector expressing green fluorescent protein (GFP)] and cell line HEK293 were used in this study.

### Example 3: Cell Culture

Cell lines were cultured and maintained in respective cell culture medium, in either T25 or T75 flasks (Cat no: 82051-074 and 82050-856, Greiner Bio-One, Monroe, NC), at 37° C. and humidified 5% CO2 in air atmosphere. All culture media were made complete by addition of Fetal Bovine Serum (PBS, final concentration 10%; Cat no: F2442-500ML, Sigma-Aldrich, St. Louis, MO) and Antibiotic-Antimycotic solution (final concentration 1×; Cat no: 15240-062, Life Technologies, Carlsbad, CA). For coronavirus studies, ST cells were cultured in complete Eagle's Minimum Essential Medium (EMEM, ATCC® 30-2003™). HEK293 cells were cultured in complete Dulbecco's Modified Eagle's Medium (DMEM, Cat no: 11995073, Gibco™, Gaithersburg, MD). For sub-culturing HEK293 cells, culture medium was discarded from flasks and cells (85-90% confluent) were rinsed briefly with 5 ml of 1× phosphate buffered saline (PBS; Cat no: 20012027, Gibco™, Gaithersburg, MD) to remove all traces of serum. Cells were detached by adding of 2-3 ml of 0.05% Trypsin-EDTA solution (Cat no: 25300054, Gibco™, Gaithersburg,

MD) and incubation at 37° C. for 15 minutes. Respective complete growth medium (˜4-6 ml) was added to the flasks. Detached cells were aspirated with gentle pipetting and this cell suspension was centrifuged at 500×g for 3 mins at 28° C. (Beckman Coulter Allegra x-14r-SX4750). Post centrifugation, supernatant was discarded, and cells were thoroughly re-suspended in either 5 ml (for T25 flask) or 12 ml (for T75 flask) of complete growth medium followed by addition in new culture flasks and incubation as mentioned earlier. For cryopreservation, cells were trypsinized and pelleted as above to re-suspend in 2 ml of complete growth medium with 5% (v/v) dimethyl sulfoxide (Cat no: BP231-100, Fisher Scientific, Waltham, MA). These cells were first stored at −20° C. for 3 h, followed by storage at −80° C. for 24 h and final storage in liquid nitrogen.

### Example 4: Respiratory Coronavirus (CoV) Infection and Propagation

ST cells were cultured in complete EMEM till they attained a confluency of 80-90% followed by washing monolayers with 5 ml of 1×PBS. USDA permit 141794 was obtained for the procurement and laboratory use of coronavirus. Coronavirus stock (ATCC VR-2384) was thawed at 37° C. for 5 min and aliquots of 250 μl were prepared for further use or storage in liquid nitrogen. An aliquot of this stock was diluted with 3 ml of incomplete culture medium (without PBS and Antibiotic-Antimycotic solution) to attain a Multiplicity of Infection (MOI) of 1 as per ATCC recommendations. This diluted viral stock was added to the washed monolayer and incubated at 37° C., humidified 5% CO₂ in air atmosphere. Flasks were rocked gently for 2 min at intervals of 30 min, to re-distribute viral inoculum. Post 2 h infection, viral adsorption was ended by adding 10 ml of complete culture medium to the monolayer. Cells were monitored microscopically every 24 h for signs of cytopathic effects (CPE). Flasks showing CPE in 80% of the infected host cells were used for viral purification.

### Example 5: Coronavirus Purification

Culture medium from flasks with infected cells was harvested at 10000×g for 20 mins at 4° C. (Beckman Coulter Allegra x-14r-FX6100). Viral precipitation from this supernatant (12 ml) was done by addition of polyethylene glycol (PEG-6000, final concentration 10%; Cat no: 81260, Sigma-Aldrich, St. Louis, MO) and NaCl (final concentration 2.2%; Cat no: 5271, Fisher Scientific, Waltham, MA). PEG-6000/NaCl/culture supernatant mixture was incubated at 4° C. for 30 mins. PEG precipitated proteins and virions were pelleted at 10000×g for 30 min at 4° C. and the pellet was dissolved in 100 μl of ice-cold 1× HEPES-saline buffer (10 mM HEPES-Sigma H7523+0.9% w/v NaCl, pH 6.7). Dissolved pellet was then loaded on a discontinuous sucrose gradient (10-20-30%, 800 μl each; freshly prepared in 1× HEPES-saline) and subjected to ultracentrifugation at 100000×g for 90 mins at 4° C. (Beckman Coulter Optima MAX-XP Ultracentrifuge). Post ultra-centrifugation, the supernatant was discarded, and the viral pellet was dissolved in 100 μl of 1× HEPES-saline buffer (pH 7.4). Total viral particle number estimation was performed using Nanoparticle Tracking Analysis (NTA) and purified viruses were flash frozen in dry ice followed by storage in liquid nitrogen, till further use.

### Example 6: Nanoparticle Tracking Analysis

Viruses were diluted in EMEM (ATCC® 30-2003TM) or 18.2 MΩ water. Mean particle diameter and concentration of viral particles were analyzed by NanoSight NS300 with a 532 nm laser and SCMOS camera (Malvern, Worcestershire, UK). Briefly, samples were diluted 100:1 or as needed in fresh milliQ to obtain 5-100 particles/frame. Samples were typically analyzed using 5 runs of 30 s collecting 25 frames per second (749 frames per run) with viscosity determined by the temperature and camera level highest available for sample (typically 15 or 14). The syringe pump speed was 60. NTA automatically compensates for flow in the sample such that only Brownian motion is used for size determination. For processing results, the detection threshold was typically 5 with automated blur size and max jump distance. Standard 100 nm latex spheres were run at 1000:1 dilution in milliQ to test optimal instrument performance Data were analyzed usung NTA 3.0 software (Malvern Instruments Ltd., UK).

### Example 7: Zeta Potential Analysis

ζ potential measurement of viral particles was determined by Zetasizer (Nano-Z, Malvern Instruments Ltd., UK). All samples were dispersed in double-distilled water and tested in volume-weighted size distribution mode in folded capillary cells (Fisher Scientific NC0491866). An average of three readings (˜60 s) were recorded.

### Example 8: Scanning Electron Microscopy of CoV

Viral particles were suspended in ddH2O with 2.5% glutaraldehyde or other buffer and dropped onto clean silica wafers. After drying, samples were desiccated in a vacuum chamber for at least 12 h before analysis. Images were obtained after carbon sputter coating using a field emission scanning electron microscope (JEOL 7800F, JEOL Japan) at a beam energy of 5 or 10 kV. For the SEM images of the fabric, gold sputter coating was used.

### Example 9: Energy Dispersive X-ray Microanalysis

For elemental detection, the Energy Dispersive X-ray (EDX) microanalysis associated to scanning electron microscopy was used. When the electron beam hits the gold sputtered fabric, some atoms of the sample are excited or ionized. When excited or ionized atom return to their ground state, they emit characteristic x-rays. The x-ray emissions at different wavelengths were measured using a photon-energy-sensitive detector. The EDX detector system performs a simultaneous display of all mid-energy (1-20 keV) x-rays collected during any individual analysis period.

### Example 10: Coronavirus and Lentivirus Infectivity

ST (coronavirus) and HEK 293 (lentivirus) cells were seeded at densities of 10,000/well and 1000/well in 24-well and 96-well cell culture plates, respectively. Seeded plates were incubated at 37 ° C., 5% CO2 humidified incubator for 18 h. One hundred microliter (10⁵ particles) of aqueous suspension of viruses (10⁶/ml of VR-2384 and CSCGW mut6 lentivirus) were spotted on 1.5 cm diameter discs of fₑ and fₛ at room temperature. After an incubation period of 1 min or 5 min, 100 μl of serum free medium was used to rinse each fabric for recovering viral particles from the fabric. NTA was performed, as above, to estimate viral recovery efficiency. Recovered VR-2384 viruses were diluted with serum free medium and used to infect ST cells at MOI of 10 (10⁵ viruses). Recovered CSCGW mut6 virus was diluted in complete DMEM medium followed by HEK293 transduction at MOI of 10 (4×10⁴ viruses). Parallel sets of cells infected with untreated viruses (at the same MOI as that of treated viruses) were used as positive control while uninfected or non-transduced host cells were accounted as negative control. Virus infected ST cells were monitored microscopically at intervals of 24 h for the onset and progression of cytopathic effects. The expression of GFP in transduced HEK293 was assessed after 4 days to ascertain the effect of fₑ treatment on lentiviral infectivity. Six technical replicates were assayed for each experimental group. Twelve biological replicates were studied.

### Example 11: Cell Viability Staining by Calcein AM and Propidium Iodide

Viability of ST cells, infected as above, was assessed by dual staining with Calcein AM and Propidium iodide (PI). Media from wells with ST cells (uninfected or infected with untreated or fabric-contacted viruses) was washed briefly with 1 ml of 1×PBS (per well) for 1 min, followed by addition of 250 μl of freshly-prepared staining solution in 1×PBS (Calcein AM; final concentration 1 μg/ml, Catalog no: C1430, Invitrogen™, Waltham, MA) and PI (final concentration 10 μg/ml, Catalog no: CAS 25535-16-4, Sigma-Aldrich, St. Louis, MO). Cells were incubated under dark conditions at 37° C. for 15 min and then observed under a Confocal Laser Scanning Microscope using a 10× objective. Multiple images (10 images per group/per set) were captured and fluorescence intensities were calculated from these images using Zen blue software and graphically plotted. The ratio of PI:Calcein signal was normalized with the average PI intensity of untreated cells to obtain fold-change compared to non-viable cells (basal cell death) in untreated cells.

### Example 12: Cell Viability Assessment by MTT Assay

Cell viability of ST cells infected as above was assayed using the MTT (3-(4,5-Dimethylthiazol-2-yl)-2,5-diphenyltetrazolium bromide) assay. This assay was performed as per manufacturer's protocol (MTT assay kit, Catalog no: ab211091, Abcam, Cambridge, MA). ST cells were washed with 1 ml of 1×PBS per well and then harvested using a cell scraper. Cells were collected in tubes, centrifuged at 300×g for 5 min at room temperature followed by re-suspension in incomplete EMEM. Cells in this suspension were counted and cell count in all the experimental groups was normalized to 10⁵ cells per 500 μl of culture medium. In separate 96-well tissue culture plates, 50 μl of the above suspension was added in each well (final cell counts as 10⁴ cells). To each well, 50 μl of MTT solution was added and the plates were incubated at 37° C., 5% CO₂ humidified incubator for 3 hours. After incubation, plates were centrifuged at 300×g for 5 min at room temperature and supernatant was discarded. One hundred and fifty microliters of MTT solvent was added to each well. Plates were wrapped in aluminum foil and rocked on an orbital shaker for 15 min followed by measurement of absorbance at 590 nm.

### Example 13: Statistical Analysis

GraphPad Prism (GraphPad Software) v8.0 was used for statistical analyses. Statistical analysis between multiple groups were performed using one-way analysis of variance with the post hoc Sidak multiple comparison test. Statistical analysis between two groups was performed using unpaired Student's two-sided t tests. P<0.05 was considered statistically significant. Significance levels and exact P values are indicated in all relevant figures. Data were normally distributed. Data for independent experiments were presented as means±SEM unless otherwise stated. Individual data points are plotted reflecting n (8-19) for each experiment.

### Example 14: Characterization of the Electroceutical Fabric

The electroceutical fabric tested is made up of polyester fabric printed with alternating circular regions of Ag and Zn dots. The Ag dots (2 mm) and Zn dots (1 mm) were printed on the fabric in proximity of about 1 mm to each other. Scanning electron microscopy (SEM) displayed the deposition of Ag particles and Zn on the fibers of the polyester fabric. Energy Dispersive X-ray (EDX) microanalysis revealed the presence of Ag and Zn on the electroceutical fabric (fₑ) and absence in the sham polyester fabric (fₛ). The only peak that was present other than C and O was that of Au used for coating the fabrics for SEM imaging. Proximity of Ag and Zn on polyester fabric forms a redox couple and is capable of driving electrochemistry when wet in an aqueous ionized environment including any body fluid. Ag and Zn were spotted on another textile which was also appropriate for the preparation of stretchable face-masks. SEM of the fabric used for such mask showed a different weaving pattern aimed at higher stretch property.

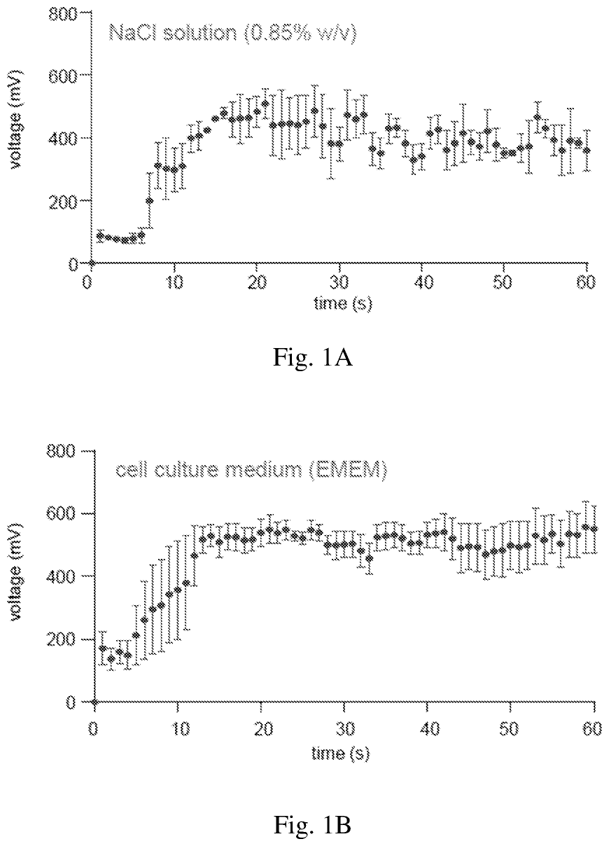

Deposition of Ag and Zn on the fabric for face-mask was tested by EDX spectrum analysis. Our primary line of investigation focused on the polyester-based electroceutical fabric which may be utilized for the development of PPE. Three ionized aqueous media were used to test potential difference between adjacent Ag and Zn deposits. NaCl solution (0.85% w/v), cell culture medium and tap water (of practical value to end users of PPE) were tested at room temperature. The potential difference between the two electrodes rapidly increased and achieved a steady state after the first 15 s (FIGS. 1A-1C).

### Example 15: Physical Characterization of the Coronavirus

SEM (150,000×) revealed the morphological features of the CoV particle. Following spotting on the silicon wafer, the purified virus was fixed and subsequently dehydrated. A thin (2-5 nm) layer of carbon was sputtered on the sample to make the specimen conductive. The size of the virus ranged between 75-125 nm. Nanoparticle tracking analysis (NTA) revealed poly- dispersed peak. The electrokinetic property, as represented by the zeta (ζ) potential, of the viral particles is a parameter that determines adsorption and stability of the particle in any given dispersant medium. For practical purposes, viral particles are expected to be suspended in water droplets either aerosolized or resting on a surface. The average ζ potential of four different preparation of CoV was determined to be −25.675 mV. All four-preparation demonstrated comparable ζ potential distribution and phase shift. The average electrophoretic mobility distribution was determined to be −2 μmcm/Vs.

### Example 16: Electroceutical Fabric Attenuated the ζ Potential of Coronavirus Upon Contact

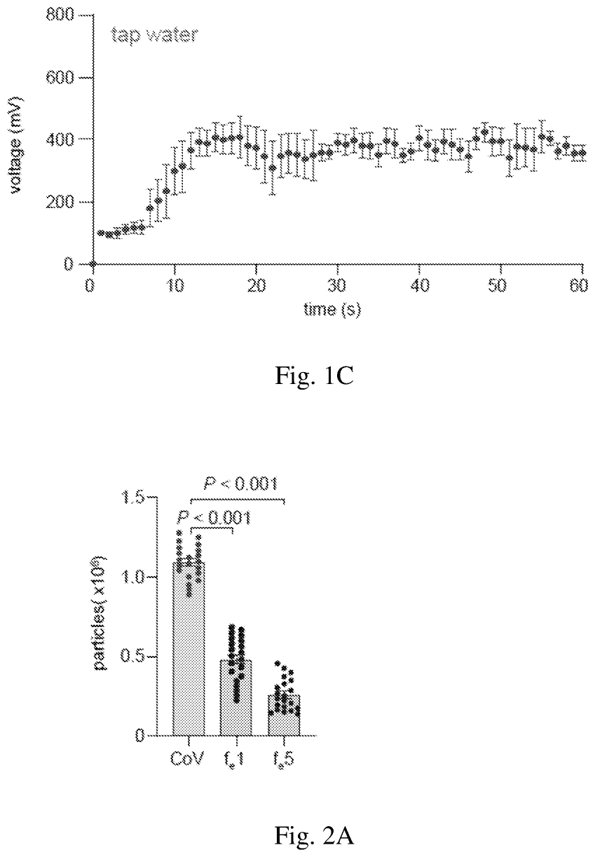

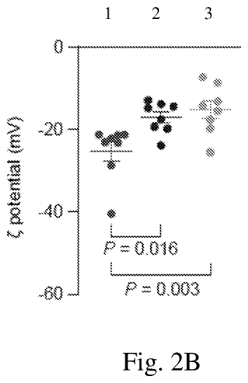

Quantification of the purified viral particles after spotting on fₑ yielded 44.29% and 23.73% recovery from the fabric when exposed for 1 min or 5 min, respectively (FIG. 2A). Nanoparticle tracking analysis demonstrated that unlike the purified CoV that showed a single peak around 75 nm, the recovered CoV showed additional peaks suggesting aggregation of the viral particles upon contact with the fabric. Analysis of ζ potential showed significant graded attenuation of this electrokinetic property upon contact with the fₑ. Such lowering of average ζ potential of CoV, applied and recovered from fₑ, has been plotted graphically (FIG. 2B). Unlike 1 min exposure to the fₑ, 5 min exposure showed an appreciable difference in the phase plot of the viral particles.

### Example 17: Loss of Corona Virus Infectivity upon Contact with Electroceutical Fabric

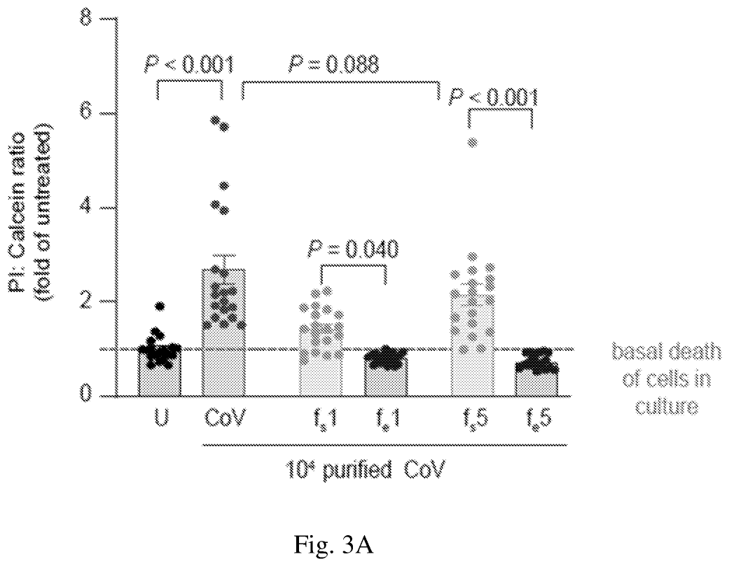

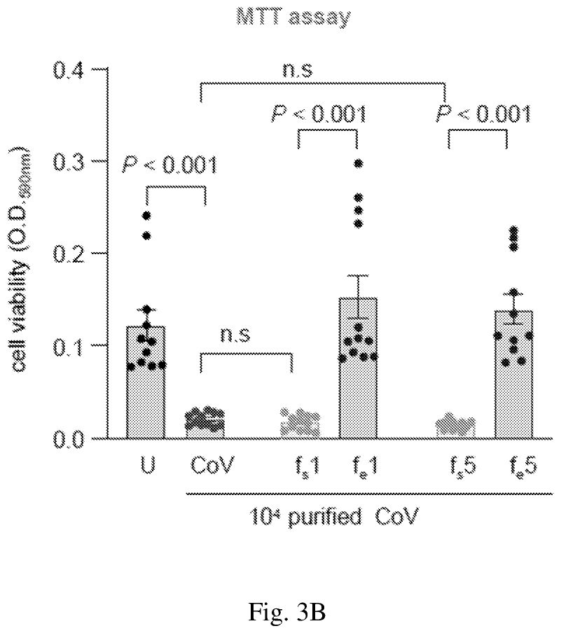

To assess changes in the infectivity of CoV following contact with the electroceutical fabric, a cytopathic assay was employed. Infected cells were monitored for appearance of cytopathic effects (CPE; cell rounding and sloughing) until post-infection day 7. Overt CPE was observed on day 7 in response to CoV infection. Comparable CPE was noted in response to treatment of cells with CoV recovered from sham control fabric fₛ. In contrast, CoV recovered from fₑ did not cause any CPE indicating loss of its infectivity. Cells treated with fₑ-recovered CoV particles appeared as healthy as the uninfected cells. Objective assessment of cell viability was performed using a calcein/PI fluorescence assay. Only live cells with intracellular esterase activity hydrolyze the acetoxymethyl ester in non-fluorescent Calcein AM converting it into green fluorescent Calcein. Dead cells or cells with damaged or compromised cell membranes include PI stain, which is otherwise impermeant to live cells. Fold-change increase in PI/Calcein signal as shown indicates loss of cell viability in response to infection. Infection of cells with CoV caused marked loss of cell viability. Such cytopathic effect of CoV was completely absent once the virus was exposed to fₑ (FIG. 3A). The sham fabric did not afford such protection (FIG. 3A). The cytopathic effects of CoV and the protective effects of fₑ (versus fₛ) was corroborated by the standard MTT assay commonly used for testing cell viability (FIG. 3B).

### Example 18: Electroceutical Fabric Eliminated Lentiviral Transduction Efficacy

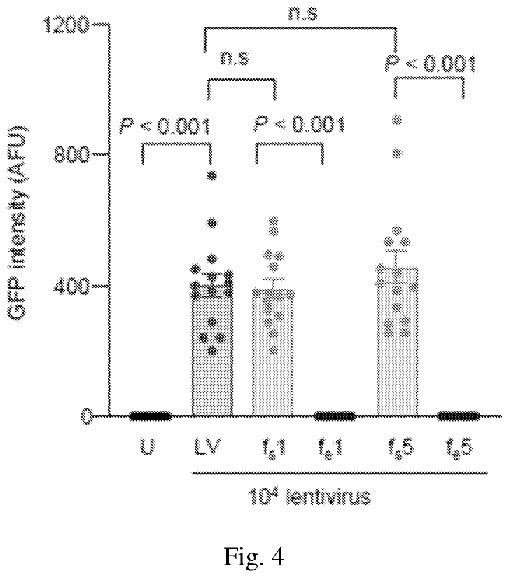

The Lentiviral pseudotype system is a standard laboratory tool to study the infectivity of viruses under conventional biosafety conditions. Lentivirus CSCGW mut6, upon successful transduction in HEK293 cells, results in GFP-expressing host cells. This expression is a direct measure of lentiviral replication competency and ability of the virus to integrate in the host genome. The ability of the electroceutical fabric to influence the infectivity of a virus, other than CoV, was tested to appreciate its broader significance of scope. Mammalian cells were treated with purified lentivirus or the same virus subjected to contact with fₑ or fₛ for 1 or 5 mins as indicated in the figure legend (FIG. 4). Transduced cells were monitored microscopically to check the presence of GFP⁺ cells, a marker of successful infection. Lentiviral exposure caused widespread infection of cells. Treatment of cells with virus recovered from sham fabric fₛ caused comparable infection (FIG. 4). However, contact of virus with the electroceutical fabric fₑ, even for one minute, eliminated lentiviral infectivity (FIG. 4).

1. A method of inactivating an RNA virus, said method comprising contacting the virus with an electroceutical material in the presence of an aqueous solution, wherein the electroceutical material comprises a plurality of silver and zinc regions that are imprinted on said material and are spaced from each other at a distance that upon contact with said aqueous solution generates an electric field or current.

2. The method of claim 1 wherein said contact results when air comprising said virus is drawn across, or through, said electroceutical material in the presence of an aqueous solution.

3. The method of claim 2 wherein the electroceutical material is part of a filtration device.

4. The method of claim 2 wherein the electroceutical material comprises a fabric of synthetic fibers having 1-2 mm sized particles of silver and zinc printed in an alternating pattern on the fabric within about 0.5 to 1.5 mm proximity of each other.

5. The method of claim 3 wherein the electroceutical material generates a potential difference of 0.5V upon contact with an aqueous solution.

6. The method of claim 4 wherein the synthetic fibers are elastic, optionally wherein the synthetic fiber comprises a polymer selected from the group consisting of polyurethane, polytrimethylene terephthalate and polybutylene terephthalate fiber.

7. The method of claim 6 wherein the electroceutical material is a component of a facial mask or screen.

8. The method of claim 2 wherein the virus is a coronavirus.

9. An air filtration system for reducing the spread of an RNA virus, comprising an electroceutical material that comprises a material having a plurality of silver and zinc regions imprinted on a surface of the material and spaced from each other at a distance that upon contact with said aqueous solution generates an electric field or current.

10. The air filtration system of claim 9 wherein the electric field or current generated by the electroceutical material disrupts the electrokinetic properties of a virus placed in contact with the electroceutical material.

11. The air filtration system of claim 10 wherein the virus is a coronavirus.

12. The air filtration system of claim 11, wherein the electroceutical material is part of an article of personal protective equipment.

13. The air filtration system of 12 wherein the personal protective equipment is a face mask.

14. The air filtration system of 13, wherein the silver and zinc is imprinted on the outer exposed surface of the facemask.

15. The air filtration system of claim 14, wherein the electroceutical fabric is designed to generate a potential difference of about 0.25 to about 1.0 V.

16. A method of disrupting the electrokinetic properties of an RNA virus comprising:
contacting the virus with an electroceutical material in the presence of an aqueous solution, wherein the electroceutical material comprising a plurality of silver and zinc regions that are imprinted on said material and are spaced from each other at a distance that upon contact with said aqueous solution generates an electric field or current that disrupts the electrokinetic properties of a the RNA virus.

17. The method of claim 16 wherein the RNA virus is a coronavirus.

18. The method of claim 16, wherein the electroceutical material generates a potential difference of about 0.25 to about 1.0 V.
